(19) United States
(12) Patent Application Publication
(10) Pub. No.: US 2025/0185900 A1
(43) Pub. Date: Jun. 12, 2025

(54) ROTATIONAL SUCTION VALVES

(71) Applicant: Boston Scientific Scimed, Inc., Maple Grove, MN (US)

(72) Inventors: Ryan Vincent William POLLOCK, Sterling, MA (US); Andrew USACHEV, Iowa City, IA (US); Julia VEITCH, Waltham, MA (US); Brian LUIS, Boylston, MA (US); Colby HARRIS, Norfolk, MA (US)

(73) Assignee: Boston Scientific Scimed, Inc., Maple Grove, MN (US)

(21) Appl. No.: 18/971,239

(22) Filed: Dec. 6, 2024

Related U.S. Application Data
(60) Provisional application No. 63/607,259, filed on Dec. 7, 2023.

(51) Int. Cl. A61B 1/015; F16K 5/08

(52) U.S. Cl. CPC A61B 1/015; F16K 5/08

(57) ABSTRACT

Systems and methods for a medical valve are disclosed. For example, the medical valve may include a valve body including a body outlet aperture and a body inlet aperture, and an inner shaft including an inner shaft outlet aperture and an inner shaft inlet aperture, wherein the inner shaft is configured to rotate such that the medical valve transitions to an active state whereby the body inlet aperture and the inner shaft inlet aperture are substantially in alignment.

### CROSS-REFERENCE TO RELATED APPLICATIONS

This application claims the benefit of priority to U.S. Provisional Application No. 63/607,259, filed on Dec. 7, 2023, which is incorporated by reference herein in its entirety.

### TECHNICAL FIELD

This disclosure relates generally to valves for medical devices, and, in particular, to suction valves for endoscopes.

### BACKGROUND

Medical devices, such as endoscopes, may include a handle portion and an insertion portion (e.g., a shaft). The insertion portion may be configured to be inserted into a body lumen of a subject. A distal tip of the insertion portion may include various elements that are controlled by the handle portion. For example, an endoscope or other medical device may include functionality to deliver fluids (including air and/or water) and suction at a site of a procedure. Conduits for delivering fluids and/or suction may extend from a handle of the endoscope, through a sheath of the insertion portion of the endoscope, and to a distal tip of the endoscope. Valves may be disposed in the handle portion in order to control delivery of the air, water, and/or suction to or from the distal tip via the conduits. For example, a suction valve may control a flow of suction through the endoscope, and a combined air/water valve may control a flow of air and water through the endoscope.

### SUMMARY

Each of the aspects disclosed herein may include one or more of the features described in connection with any of the other disclosed aspects.

Aspects of the disclosure relate to a medical valve that may include a shaft including a first opening, a second opening, and an actuator.

The first opening may be on a distal end of the shaft and the second opening may be on a side surface of the shaft.

The actuator may include a rotatable knob or a rotatable lever. The actuator may include a linear actuator configured to move in a direction parallel to the longitudinal axis of the shaft. The linear actuator may include a button. The actuator may be configured to automatically transition from the first configuration to the second configuration. The actuator may be configured to rotate the shaft about a longitudinal axis of the shaft in order to transition the valve to (a) a first configuration, in which the second opening is aligned with a port of a valve well, in order to deliver suction to a working channel of an endoscope, and (b) a second configuration in which the second opening is misaligned with the port of the valve well.

The shaft may include a protrusion configured to engage with a groove of the button. The shaft may be an inner shaft. The medical valve may further comprise an outer shaft. The outer shaft may include a third opening on a distal end of the outer shaft and a fourth opening on a side surface of the outer shaft. In the first configuration, the second opening of the inner shaft may be aligned with the fourth opening of the outer shaft. The fourth opening may be aligned with the port in the first configuration and the second configuration.

The outer shaft may include a channel on the side surface of the outer shaft. The channel may be aligned with the second opening of the inner shaft in the second configuration, such that the first opening of the inner shaft is in fluid communication with the channel of the outer shaft. The channel may be in fluid communication with an atmosphere in at least the second configuration. The outer shaft may include a cut or groove. The inner shaft may include a protrusion configured to engage the cut or the groove.

The actuator may be fixedly coupled to the inner shaft, such that linear movement of the actuator causes the protrusion of the inner shaft to ride along the cut or the groove in order to rotate the inner shaft relative to the outer shaft. The inner shaft may include a fifth opening. In the second configuration, the fifth opening may be proximal of a proximal-most end of the outer shaft and the valve well.

The shaft may include an outer surface that may include a recessed portion. The recessed portion may be aligned with the port in the second configuration, such that the port is in fluid communication with an atmosphere in the second configuration. The recessed portion may be angularly offset from the second opening by at least approximately 90 degrees. The medical valve may further comprise an aperture. The aperture may be configured to be closed in the first configuration and open in the second configuration.

In another example, the medical valve may comprise a threaded shaft and a rotatable body. The threaded shaft may define a shaft lumen extending longitudinally through at least a portion of the threaded shaft.

The rotatable body may be configured to rotate with respect to the threaded shaft in order to selectively align the spoke lumen with a port of a suction valve well. The rotatable body may include at least one spoke. The at least one spoke may define a spoke lumen. The port may be a first port. The shaft lumen may have an open distal end configured to be aligned with a second port of the suction valve well in all configurations of the medical valve.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction extending away from an operator (e.g., toward or further into a body lumen of a subject), and the term “proximal” refers to the opposite direction, toward the operator (e.g., away from an interior of a subject's body). Some drawings include arrows labeled “proximal” and “distal” to indicate proximal and distal directions, respectively. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIGS. 1A-1B depict an exemplary medical device.

FIG. 2 shows a cross-sectional side view of a handle assembly of a medical device according to some embodiments.

FIGS. 3A-3C show a first exemplary valve assembly.

FIGS. 4A-4D show a second exemplary valve assembly.

FIGS. 5A-5C show a third exemplary medical valve assembly.

FIGS. 6A-6C show a fourth exemplary valve assembly.

FIGS. 7A-7B show a fifth exemplary valve assembly.

### DETAILED DESCRIPTION

A medical valve of a medical device (e.g., an endoscope) may be configured to control delivery of suction during a procedure. To delivery suction, the valve may place a working channel of the medical device (or another lumen of the medical device) in fluid communication with a source of suction. Conventional suction valves include a valve stem that is translated within a valve well of a handle of the medical device. Such valves may become stuck and fail to return completely to the resting state in order to turn off a flow of suction. The disclosed embodiments transition from an active state (transmitting a flow of suction) to a resting state (inhibiting the flow of suction) via non-translational movement. In embodiments, the disclosed valves may be a single-use valve, and therefore disposable after only one procedure and post-procedure, although in other embodiments the valves may be reusable. In a first embodiment, a valve may be configured to rotate to selectively place a lumen of the valve in communication with an opening of the valve well, to allow a flow of suction through the opening. In a second embodiment, the valve may include a lever that is selectively rotatable to selectively allow a flow of suction. In a third configuration, the valve may include a threaded shaft, along which a rotating piece may travel to selectively place a source of suction in fluid communication with the working channel of the medial device. In a fourth configuration, the valve may be include a contoured rotatable shaft that selectively places a suction source in fluid communication with the working channel. In a fifth configuration, the valve may include a button that is configured to translate, thus rotating an inner shaft to selectively place the suction source in fluid communication with the working channel.

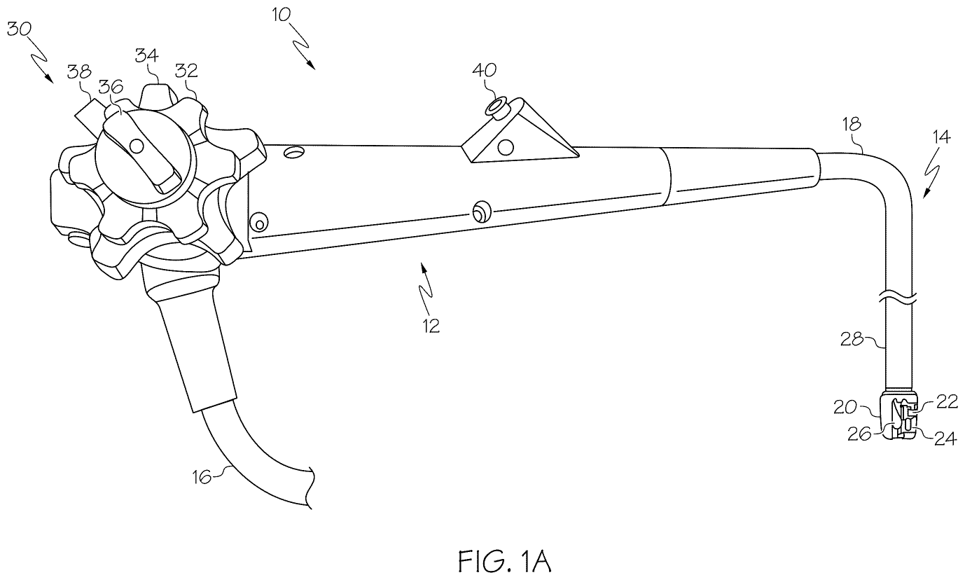

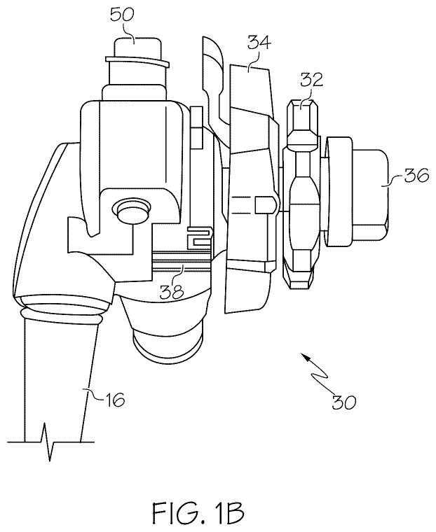

FIG. 1A depicts an exemplary medical device 10 having a handle 12 and an insertion portion 14. FIG. 1B shows a proximal end of handle 12. Medical device 10 may also include an umbilicus 16 for purposes of connecting medical device 10 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 18 and a distal tip 20. Distal tip 20 may include an imaging device 22 (e.g., a camera) and a lighting source 24 (e.g., an LED or an optical fiber). Distal tip 20 may be side facing. That is, imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device 22 may face distally (approximately parallel to a longitudinal axis of distal tip 20/shaft 18).

Distal tip 20 may also include an elevator 26 for changing an orientation of an accessory device or a tool inserted in a working channel of medical device 10. Elevator 26 may alternatively be referred to as a swing stand, pivot stand, raising base, or any suitable other term. Elevator 26 may be pivotable via, e.g., an actuation wire or another control element that extends from handle 12, through shaft 18, to elevator 26.

A distal portion of shaft 18 that is connected to distal tip 20 may have a steerable section 28. Steerable section 28 may be, for example, an articulation joint. Shaft 18 and steerable section 28 may include a variety of structures which are known or may become known in the art.

Handle 12 may have one or more actuators/control mechanisms 30. One or more of control mechanisms 30 may provide control over steerable section 28. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36 (e.g., knobs or levers) for preventing steering and/or braking of steerable section 28 in at least one of an up, down, left, or right direction. Handle 12 may include an elevator control lever 38 (see FIG. 1B). Elevator control lever 38 may raise and/or lower elevator 26, via connection between lever 38 and an actuating wire that extends from lever 38, through shaft 18, to elevator 26. A port 40 may allow passage of a tool through port 40, into a working channel of the medical device 10, through shaft 18, to distal tip 20.

Handle 12 may also include one or more valves 50. For example, valve(s) 50 may be used deliver air/water and/or suction. For example, one of valve(s) 50 may selectively deliver air and/or water to distal tip 20. Another of valve(s) 50 may selectively place the working channel of device 10 in fluid communication with a source of suction, such that a negative pressure is applied at distal tip 20.

In use, an operator may insert at least a portion of shaft 18 into a body lumen of a subject. Distal tip 20 may be navigated to a procedure site, e.g., a target site, in the body lumen. The operator may insert an accessory device (not shown) into port 40, and pass the accessory device through shaft 18 via a working channel to distal tip 20. The accessory device may exit the working channel at distal tip 20. The user may use elevator control lever 38 to raise elevator 26 and angle the accessory device toward a desired location (e.g., a papilla of the pancreatico-biliary tract). The user may use the accessory device to perform a medical procedure. During the procedure, valve(s) 50 may be actuated to deliver air, water, and/or suction.

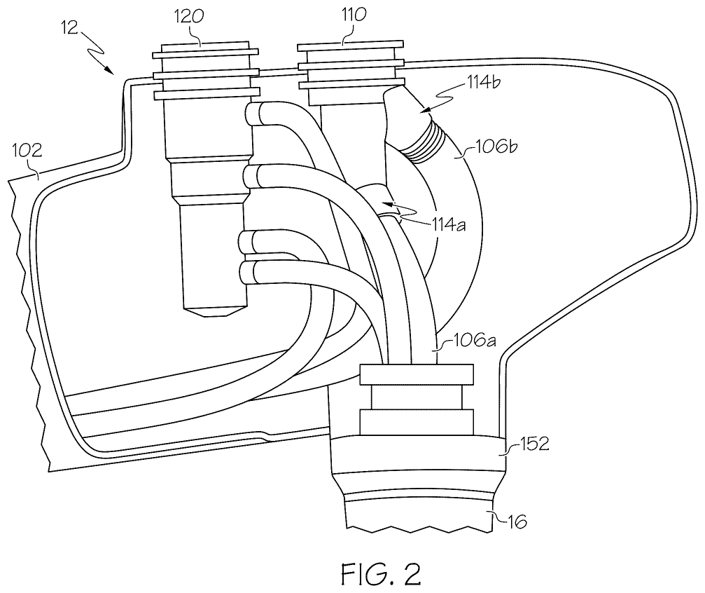

FIG. 2 shows a cross-sectional side view of handle 12 according to some embodiments. For example, handle 12 may include a handle body 102 configured to house at least inlet tubing 106a, outlet tubing 106b, a suction valve well 110. In FIG. 2, handle 12 is shown without a valve in suction valve well 110. When inserted into the suction valve well 110, the valve may function to selectively supply suction to distal tip 20. Valve well 110 may include a valve well wall 105 defining a lumen for receiving a valve therein. Handle 12 may also include an air/water valve well 120, which may receive a valve for controlling a flow of air and/or water to distal tip 20.

Inlet tubing 106a may extend from a source of suction, through an umbilicus 16, and into handle body 102 via a coupling section 152 of handle body 102 that couples handle body 102 to umbilicus 16. Inlet tubing 106a may extend through the coupling section 152 to suction valve well 110. Inlet tubing 106a may couple to an inlet port 114a of suction valve well 110. Although suction will flow proximally, the term “inlet” is used to refer to structures associated with the source of suction (e.g., a vacuum pump). Outlet tubing 106b may extend distally from suction valve well 110, through handle body 102. Outlet tubing 106b may be fluidly connected to the working channel (not shown) of device 10. The working channel may extend distally through shaft 18 to distal tip 20. Although suction will generally flow proximally through the working channel, the term “outlet” is used to refer to structures associated with the working channel.

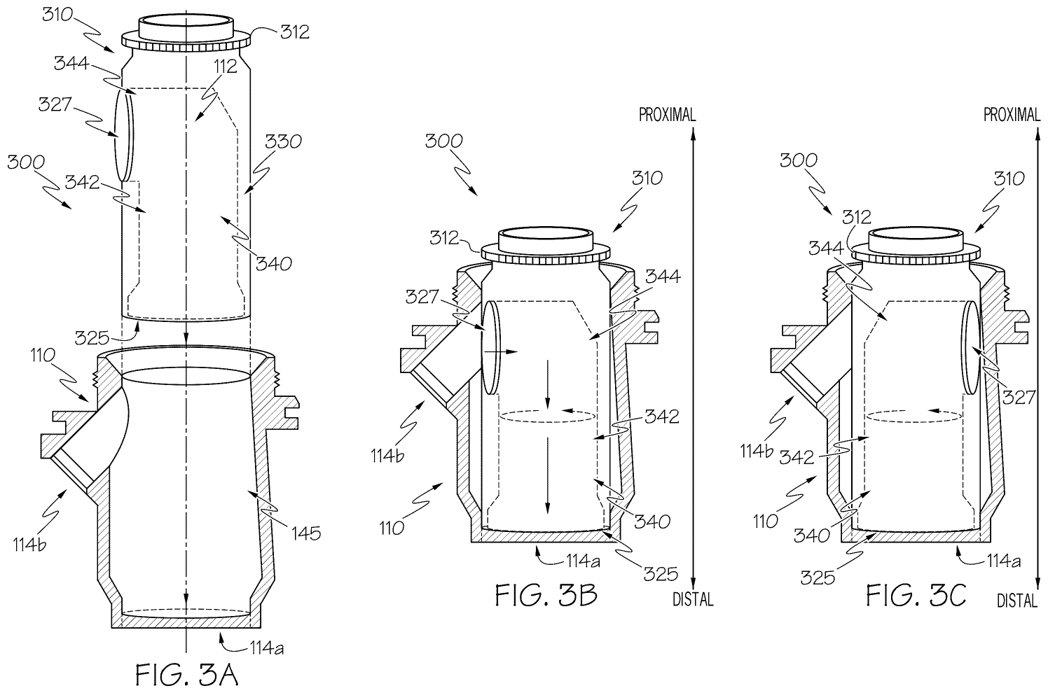

Suction valve well 110 may receive (e.g., removably receive) a suction valve (not shown in FIG. 2 and examples of which are described below). The suction valve may function to provide suction to medical device 10, for example distal tip 20, during a medical procedure via a vacuum source. The suction valve may be any of the embodiments discussed below in relation to FIGS. 3A-7B.

FIGS. 3A-3C show a first valve assembly 300. Although different exemplary embodiments are described herein, it will be appreciated that features of the exemplary embodiments may be combined. Valve assembly 300 may include a valve 310 and suction valve well 110. As discussed in further detail below, valve assembly 300 may be configured to operate by rotating valve 310 relative to suction valve well 110. Valve assembly 500 FIG. 3A shows valve 310 separated from suction valve well 110. As shown in FIGS. 3B and 3C, valve 310 may be configured to be inserted into suction valve well 110 (e.g., such that valve 310 is concentrically housed at least partially within suction valve well 110). In FIGS. 3B and 3C, valve 310 may extend from a proximal portion of suction valve well 110 to a distal portion of suction valve well 110.

Valve 310 may include an actuator 312, an inlet aperture 325, an outlet aperture 327, and an inner lumen 340 extending between inlet aperture 325 and outlet aperture 327. Actuator 312 may be disposed at a proximal end of valve 310. Actuator 312 may be coupled (e.g., fixedly coupled) to a valve stem 330 of valve 310, such that actuator 312 may enable a user to rotate valve 310 (including valve stem 330) within suction valve well 110. Actuator 312 is depicted as a knurled knob but may additionally or alternatively include a lever or any other type of knob that is suitable to rotate valve 310. In the examples depicted, actuator 312 may be similar to a dial. For example, a user may manually rotate actuator 312, causing valve 310 to rotate.

Inlet aperture 325 may be disposed on a distal end (e.g., on a distal surface) of valve 310. When valve 310 is inserted into suction valve well 110 (as shown in FIGS. 3B and 3C), inlet aperture 325 may align with inlet port 114a of suction valve well 110. In alternative where suction valve well 110 has an alternative configuration, inlet aperture 325 may disposed on other portions of valve well 310.

Outlet aperture 327 may be disposed on a radially outer side of valve stem 330 (through a radially outer wall of valve stem 330). As shown in FIGS. 3B and 3C, outlet aperture 327 may be axially aligned with outlet port 114b. Although not shown in FIGS. 3A-3B, valve stem 330 may include one or more seals thereon (e.g., around inlet aperture 325 or outlet aperture 327). The seal(s) may be any configuration and/or be comprised of any material, such as a wiper seal, an O-ring seal, rubber, polytetrafluoroethylene (PTFE), fluorosilicone (FVMQ), polyurethane (AU, EU), other polymers, etc.

Lumen 340 may extend through valve stem 330, between inlet aperture 325 and outlet aperture 327. In examples, lumen 340 may have a first, distal portion 342 and a second, proximal portion 344. Distal portion 342 may have a central longitudinal axis that is substantially aligned with a central longitudinal axis 112 of valve stem 330 and/or suction valve well 110. Proximal portion 344 may have a central longitudinal axis that is approximately perpendicular to the central longitudinal axis of distal portion 342. Inlet aperture 325 may be an opening of lumen 340 at a distal end of distal portion 342. Outlet aperture may be an opening of lumen 340 at a radially outer end of proximal portion 344. Inlet aperture 325 may be in fluid communication with outlet aperture 327 via lumen 340.

Valve 310 may be rotatable, using actuator 312, about central longitudinal axis 112 of valve 310/valve stem 330 and suction valve well 110. As valve 310 rotates, outlet aperture 327 may be selectively aligned (e.g., circumferentially aligned) with outlet port 114b, such that outlet aperture 327 is selectively in fluid communication with outlet port 114b, as described in further detail below.

FIG. 3B depicts an active state of valve assembly 300. As shown in FIG. 3B, when valve 310 is rotated (e.g., rotated around central longitudinal axis 112), such as by a user applying a shear or tangential force to actuator 312, outlet aperture 327 may be moved into alignment (e.g., circumferential alignment) or substantially in alignment with outlet port 114b. It will be appreciated that the broken arrow in FIG. 3B shows an exemplary direction of rotation but that valve 310 may alternatively be rotated in the opposite direction. For example, outlet aperture 327 may be concentric with outlet port 114b. When outlet aperture 327 is in alignment with outlet port 114b, inlet port 114a may be in fluid communication with outlet port 114b, such that a source of suction coupled to inlet port 114a may be coupled to outlet port 114b. Suction may be able to flow through inner lumen 340 via inlet aperture 325 and outlet aperture 327, and through a working channel extending through shaft 18 (see FIG. 1A). The arrows in FIG. 3B depict a direction of the flow of suction.

FIG. 3C depicts a resting state of valve assembly 300. As depicted in FIG. 3C, outlet aperture 327 may be in misalignment (e.g., may be circumferentially misaligned) with outlet port 114b. Outlet aperture 327 may be completely misaligned from outlet port 114b, such that no portion of outlet aperture 327 overlaps a portion of outlet port 114b. For example, relative to the embodiment of FIG. 3B, valve stem 330 may be rotated within shaft 145, such that outlet aperture 327 is substantially opposite (e.g., 180 degrees from or otherwise misaligned from) from outlet port 114b. Although FIG. 3C shows a position of outlet aperture 327 as being diametrically opposed from a position of outlet aperture 327 in FIG. 3B, it will be appreciated that such an arrangement is not limited; any amount of rotation sufficient to misalign outlet aperture 327 from outlet port 114b may be utilized. When outlet aperture 327 is misaligned from outlet port 114b, suction may be unable to flow through inner lumen 340 because outlet port 114b may not be in fluid communication with inlet port 114a. In both the resting state (FIG. 3C) and the active state (FIG. 3B), inlet aperture 325 may be aligned with inlet port 114a (e.g., because inlet port 114a and inlet aperture 325 extend around central longitudinal axis 112 and thus rotation of valve 310 does not alter a relative position of inlet port 114a and inlet aperture 325).

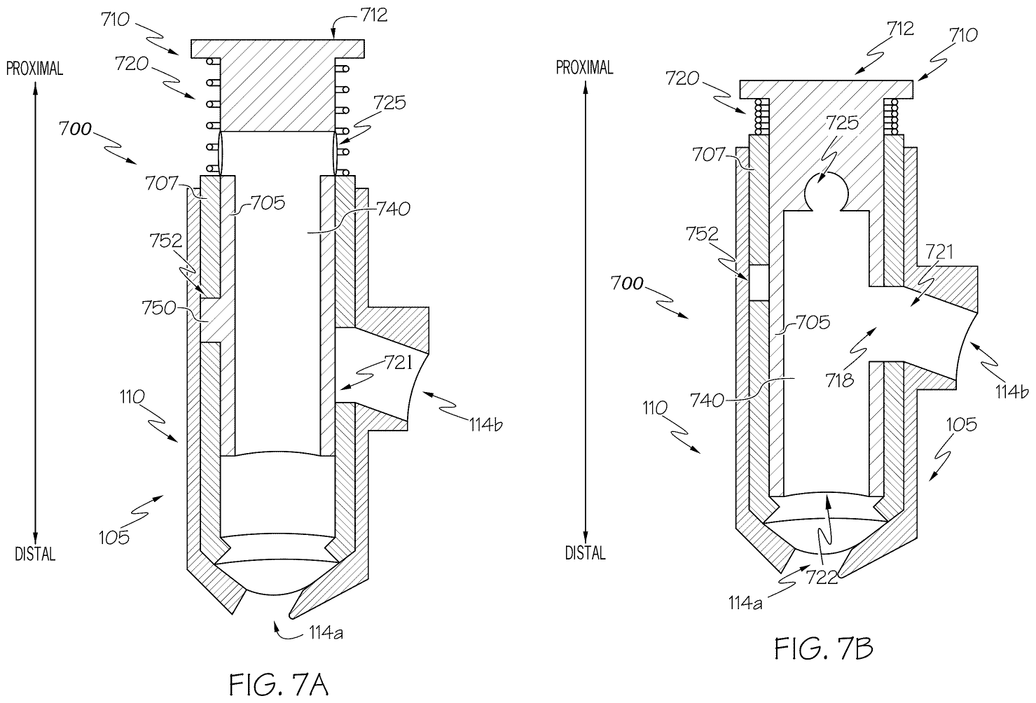

Valve assembly 300 may transition from the active state (see FIG. 3B) to the resting state (see FIG. 3C) manually or automatically. For example, a user may rotate actuator 312 in a direction opposite to the rotation that moved valve 310 to the active state, thereby changing valve assembly 300 to a resting state. In another example, valve assembly 300 may include a spring or other resilient material (see FIGS. 7A-7B) that may be configured to cause valve assembly 300 to automatically transition into the resting state upon the user removing force from actuator 312.

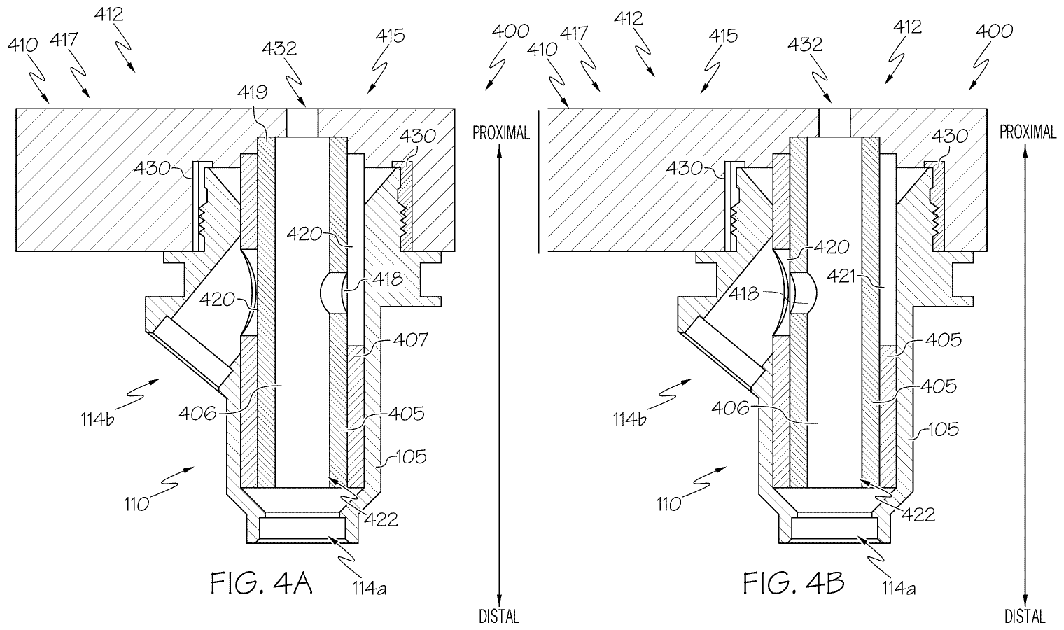

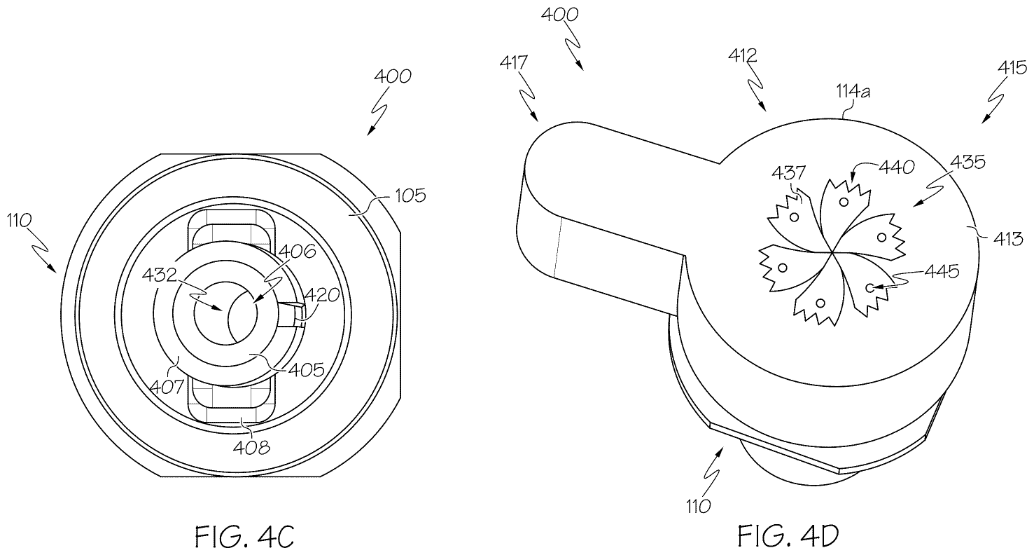

FIGS. 4A-4D show a valve assembly 400 that may be configured to operate by rotating portions of a valve 410 within suction valve well 110. Valve assembly 400 may have any of the features discussed in relation to valve assembly 500 any of the other valve assemblies described herein, unless otherwise specified.

Valve 410 may include an inner shaft 405 concentrically disposed within an outer shaft 407. Inner shaft 405 and outer shaft 407 may together form a valve stem of valve 410. A cap 412 may be disposed on a proximal portion of inner shaft 405 and outer shaft 407. Cap 412 may include an actuator 415. Valve 410 may be removably inserted into suction valve well 110. When valve 410 is inserted into suction valve well 110, inner shaft 405 and outer shaft 407 may be concentric with suction valve well 110. Valve 410 may extend from a proximal portion of suction valve well 110 to a distal portion of suction valve well 110. As discussed in further detail below, inner shaft 405 may be rotatable relative to outer shaft 407

Outer shaft 407 may be generally tubular shaped, having an outer wall and defining an inner lumen. Outer shaft 407 may be fixedly coupled to cap 412 and valve well 110 when valve 410 is inserted into valve well 110. Thus, outer shaft 407 may be configured to remain stationary during use of valve 410. Outer shaft 407 may include an aperture (hereinafter “external shaft aperture”) 420 configured to be aligned with (e.g., axially and circumferentially aligned) with outlet port 114b, such that external shaft aperture 420 is in fluid communication with outlet port 114b. One or more seals (e.g., a wiper seal or O-ring seal) or an interference fit may immovably secure outer shaft 407 relative to valve well 110.

As shown in FIGS. 4A-4C, a side of outer shaft 407 (e.g., a side opposite external shaft aperture 420 or another portion of outer shaft 407 offset from external shaft aperture 420) may include a channel 421. FIG. 4C shows valve 410 disposed in valve well 110 and with cap 412 removed to show details of outer shaft 407. Channel 421 may be a slot that extends from a proximal end of outer shaft 407 to a portion of outer shaft 407 that is distal to an outlet aperture 418 of inner shaft 405, discussed below. Channel 421 may assist in venting of suction, as discussed below.

Inner shaft 405 may be concentrically housed/disposed within outer shaft 407, and may extend from a proximal portion of outer shaft 407 to a distal portion of outer shaft 407. Inner shaft 405 may be generally tubular shaped and may define a lumen 406. In some embodiments, inner shaft 405 may extend proximally beyond the proximal-most end of outer shaft 407 and mechanically attach to actuator 415 of cap 412 at attachment point 419, as depicted in FIGS. 4A-4B and described in further detail below. Inner shaft 405 may be fixed with respect to actuator 415, such that activation of actuator 415 may cause rotation of inner shaft 405 relative to outer shaft 407, as discussed below. For example, inner shaft 405 may include one or more projections 408 (FIG. 4C) that may be coupled to actuator 415.

Inner shaft 405 may include an inlet aperture 422 (see FIGS. 4A-4B) and an outlet aperture 418. Outlet aperture 418 may be disposed on a radially outer wall of inner shaft 405 and may be axially aligned with outlet port 114b of valve well 110. Inlet aperture 422 may be disposed on a proximal end of inner shaft 405 and may align with inlet port 114a. As inner shaft 405 rotates, inlet aperture 422 may remain in fluid communication with inlet port 114a because a center of inlet aperture 422 may be coaxial with a central longitudinal axis of inner shaft 405.

As inner shaft 405 rotates about a central longitudinal axis of inner shaft 405, relative to outer shaft 407, outlet aperture may be selectively positioned in communication with outlet port 114b. That is, in some rotational configuration(s) of inner shaft 405, outlet aperture 418 may be aligned (e.g., circumferentially aligned) with outlet port 114b and external shaft aperture 420, such that outlet aperture 418 and outlet port 114b are in fluid communication. In other rotational configuration(s) of inner shaft 405, outlet aperture 418 may be offset (e.g., circumferentially offset) from outlet port 114b and external shaft aperture 420, such that outlet aperture 418 and outlet port 114b are not in fluid communication. In configurations in which outlet aperture 418 is misaligned with outlet port 114b and external shaft aperture 420, outlet aperture 418 of inner shaft 405 may be aligned with channel 421 of outer shaft 407.

In examples, inner shaft 405 may include Teflon or another self-lubricating material to prevent or inhibit leakage of suction past the interaction point(s) between inner shaft 405 and outer shaft 407. Such examples may allow for inner shaft 405 and lumen 406 to be relatively larger than embodiments with separate seals. In other examples, one or more seals may be positioned about outlet aperture 418 to inhibit or prevent suction from flowing into spaces between inner shaft 405 and outer shaft 407.

Cap 412 may include actuator 415 and a coupler 430. Coupler 430 may be configured to interact with features of suction valve well 110, such that coupler 430 is fixedly and removably coupled to suction valve well. For example, coupler 430 may include a flexible, resilient material that is designed to stretch over a proximal portion of valve well 110. Coupler 430 and valve well 110 may include complementary features (e.g., notches, grooves, or the like) for fixing, engaging, etc. coupler 430 relative to valve well 110. Alternatively, coupler 430 and valve well 110 may include threads such that coupler 430 may be screwed onto valve well 110. In such screw-on examples, coupler 430 may be constructed of rigid or flexible material. Thus, coupler 430 may couple outer shaft 407 to valve well 110 such that it is fixed with respect to valve well 110.

Actuator 415 may be rotatable relative to coupler 430. Actuator 415 may include a lever 417. A user may spin lever 417 in order to rotate actuator 415. Lever 417 may be similar to a bike bell. In the cross-sectional views of FIGS. 4A and 4B, a force exerted into or out of the page of FIGS. 4A and 4B may be applied by a user in order to rotate/spin lever 417. Although not shown, cap 412 may include one or more biased features (e.g., springs or flexible materials) such that lever 417 returns to a resting state (see FIG. 4A) when lever 417 is released by a user. Alternatively, lever 417 may be manually returned to the resting state.

Cap 412 may also include a vent 432. In some examples, a proximal portion of inner shaft 405 may be closed such that vent 432 is in direct fluid communication only with channel 421. In other examples, a top portion of inner shaft 405 may be open such that lumen 406 of inner shaft 405 is in direct fluid communication with vent 432. In some embodiments, cap 412 may include an eyelet 435 (see FIG. 4D) that may be configured to control flow through vent 432. Eyelet 435 may function similarly to a camera shutter. Eyelet 435 may include a plurality of eyelet body portions 437 (e.g., leaves or petals), a plurality of interaction points 440 (e.g., gear teeth), and a plurality of hinges 445. Rotation of lever 417 may cause a rotation of a body 413 of cap 412. Through direct or indirect interaction between body 413 and interaction points 440, rotation of gear body 413 may induce eyelet body portions 437 to pivot about hinges 445 to open and/or close. Eyelet 435 is shown in a closed configuration in FIG. 4D. In the closed configuration, eyelet 435 may prevent or inhibit flow through vent 432. In the open configuration (not shown), eyelet 435 may permit flow through vent 432. As discussed, further below, when valve 410 is in a resting state, (FIG. 4A), eyelet 435 may be open such that valve 410 is vented and inlet port 114a is in communication with the external atmosphere. When valve 410 is in an active state (FIG. 4B), eyelet 435 may be closed. It will be appreciated that eyelet 435 may include various intermediate structures that are known in the art and not depicted in FIG. 4D for ease of illustration.

In other embodiments (e.g., examples in which a proximal end of inner shaft 405 is closed), eyelet 435 may be omitted. In embodiments having eyelet 435, channel 421 may be omitted. Valve 410 is shown as including both eyelet 435 and channel 421 for fullness of illustration. However, it will be appreciated that valve 410 may include only one or the other of eyelet 435 or channel 421.

FIG. 4A depicts valve assembly 400 in a resting state. As shown in FIG. 4A, in the resting state, outlet aperture 418 may be in circumferential misalignment with outlet port 114b and external shaft aperture 420. When outlet aperture 418 is in misalignment with outlet port 114b, outlet port 114b may not be in fluid communication with outlet port 114b, such that suction does not flow through the working channel of device 10. Outlet aperture 418 may be aligned with channel 421, which may be in fluid communication with vent 432, such that suction from inlet port 114a is vented via vent 432. In examples having an eyelet 435, eyelet 435 may be open. The resting state of FIG. 4A may be a default state of valve 410, to which valve 410 returns when a user releases a force on lever 417.

FIG. 4B depicts valve assembly 400 in an active state. As shown in FIG. 4B, when a user applies force to lever 417, outlet aperture 418 may rotate into alignment with outlet port 114b and external shaft aperture 420. When outlet aperture 418 is in alignment with outlet port 114b, suction provided by inlet port 114a may be able to flow through the working channel shaft 18 (see FIG. 1A) via outlet port 114b. Outlet aperture 418 may be misaligned with channel 421 in the active state. In some embodiments including eyelet 435, when valve assembly 400 is in the active state (see FIG. 4B), eyelet 435 may be closed.

Valve assembly 400 may transition from the active state (see FIG. 4B) to the resting state (see FIG. 4C) manually or automatically. For example, a user may rotate or release lever 417, thereby changing valve assembly 400 to a resting state. In another example, valve assembly 400 may include a spring or other pliable material that may be configured to cause valve assembly 400 to transition into the resting state upon the user removing force from lever 417.

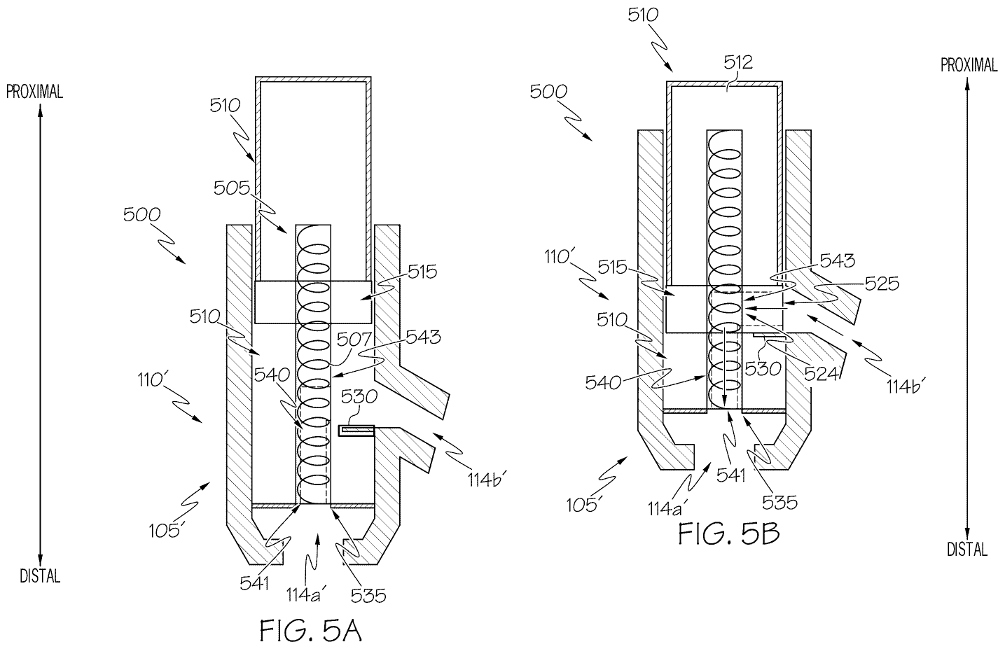

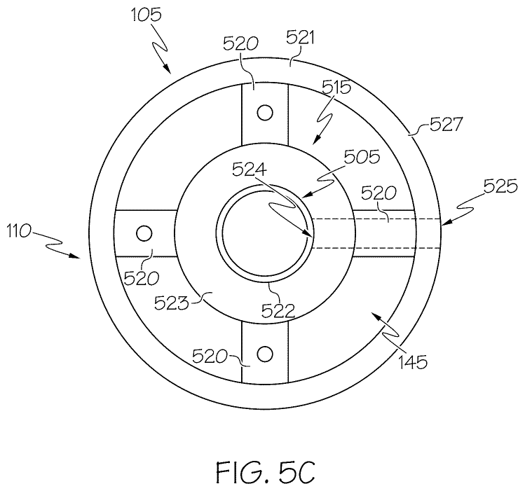

FIGS. 5A-5C show another valve assembly 500 that may be configured to operate by rotating and/or translating a movable portion 515 of a valve 510 within suction valve well 110′. FIGS. 5A and 5B show valve 510 positioned within valve well 110′. FIG. 5C shows a distal-facing view of a proximal end of valve 510. Valve assembly 500 may have any of the features discussed in relation to medical device 10, valve assembly 300, valve assembly 400, or the assemblies described below, unless provided otherwise herein. Suction valve well 110′ is depicted schematically in FIGS. 5A-5C and may have any of the properties as suction valve well 110 described above, although aspects of suction valve well 110′ may appear differently due to the schematic representation.

Valve 510 may include a shaft 505 and movable portion 515 (e.g., a rotatable body). Movable portion 515 may include a plurality of spokes 520, as depicted in FIG. 5C. Spokes 520 may extend from a central portion 523 of movable portion 515 to an outer portion 527 of movable portion 515. Central portion 523 may be annular and may extend around shaft 505. Outer portion 527 may be annular and may be concentric with central portion 523. An overall shape of movable portion 515 may be similar to a wagon wheel. At least one of spokes 520 may include a lumen 521 that may extend through spoke 520. A radially inner portion of lumen 521 may extend through an entirety of a wall of central portion 523 to an inner wall aperture 524 (e.g., a central aperture) on an inner wall of central portion 523. A radially outer portion of lumen 521 may extend through an entirety of a wall of outer portion 527 to form a spoke aperture 525 on an outer surface of outer wall portion 527.

Movable portion 515 may be configured to rotate around and with respect to shaft 505. In some embodiments, central portion 523 of movable portion 515 may include a threaded surface on an inner surface of central portion 523 (hereinafter “movable portion threaded surface”) 522. Shaft 505 may include a threaded surface on an outer surface of shaft 505 (hereinafter “shaft threaded surface”) 507 (see FIG. 5A). Shaft threaded surface 507 may be configured to pair with and interact with movable portion threaded surface 522.

Shaft 505 may include a central lumen 540 extending axially along at least a portion of a length of shaft 505, between an inlet shaft aperture 541 and an outlet shaft aperture 543. Although other portions of assembly 500 are shown in cross-section, shaft 505 is not shown in cross-section. Lumen 540 is shown in broken lines in FIGS. 5A and 5B because it is internal to shaft 505. Inlet shaft aperture 541 may be on a distal-most end of shaft 505. Outlet shaft aperture 543 may be formed in a sidewall (a radially outer wall) of shaft 505. When valve 510 is positioned in suction valve well 110′, inlet shaft aperture 541 may be aligned with/in fluid communication with an inlet port 114a′ of suction valve well 110′. A distal surface 535 of valve 510 may inhibit flow of suction along sides of shaft 505 and may permit suction to flow only via shaft 505.

An actuator 512 may be coupled to one or more of movable portion 515 or shaft 505. In some examples, shaft 505 may be axially and rotationally fixed relative to suction valve well 110′, and movable portion 515 may be axially and rotatably movable relative to shaft 505. Actuator 512 may be coupled to movable portion so as to rotate movable portion 515, thereby moving it axially (proximally or distally) along shaft 505. For example, actuator 512 may be a knob or other rotatable actuator.

Alternatively, shaft 505 may be rotationally fixed but axially movable relative to suction valve well 110′. Movable portion 515 may be axially fixed relative to suction valve well 110′. Shaft 505 may function similar to a lead screw. As shaft 505 is moved proximally or distally, movable portion 515 may rotate about an axis of shaft 505 and remain axially fixed. Actuator 512 may be coupled to shaft 505 to move it proximally or distally. For example, actuator 512 may include a button.

Alternatively, shaft 505 may be rotatably movable relative to suction valve well 510. Movable portion 515 may be rotatably fixed relative to suction valve well 110′. Thus, shaft 505 may function like a lead screw. As shaft 505 rotates, movable portion 515 may move axially, in a proximal or distal direction. Actuator 512 may be coupled to shaft 505 to rotate it. For example, actuator 512 may include a knob or other rotatable actuator.

FIG. 5A depicts valve assembly 500 in a resting state. As shown in FIG. 5A, when no force is applied to actuator 512, spoke aperture 525 of movable portion 515 may be in misalignment with an outlet port 114b′. Although movable portion 515 is shown as being axially offset from outlet port 114b′ and outlet shaft aperture 543, it will be appreciated that movable portion 515 (including lumen 521 of spoke 520) may be axially aligned with shaft aperture 543/outlet port 114b′ and radially misaligned with shaft aperture 543/outlet port 114b′. When spoke aperture 525 and/or movable portion 515 are in misalignment with outlet port 114b′, as shown in FIG. 5A, outlet port 114b′ is not in fluid communication with central lumen 540 and inlet port 114a′. Thus, in the configuration of FIG. 5A, suction does not flow through device 10. In the configuration of FIG. 5A, suction from inlet port 114a′ may pass through inlet shaft aperture 541, outlet shaft aperture 543, and may vent through a proximal portion of valve 510 (e.g., through spaces between spokes 520).

FIG. 5B depicts valve assembly 500 in an active state. As shown in FIG. 5B, when force is applied to actuator 512 (e.g., via a user applying a twisting or pushing force to actuator 512), movable portion 515, including spoke 520 having lumen 521, may move or transition into alignment with outlet port 114b′. When spoke aperture 525, lumen 521, and central aperture 524 are in alignment with outlet port 114b′ and outlet shaft aperture 543, suction may be able to flow from inlet port 114a′, through inlet shaft aperture 541, central lumen 540, outlet shaft aperture 543, central aperture 524, lumen 521 of spoke 520, spoke aperture 525, outlet port 114b′, and the working channel of device 10. Where movable portion 515 is axially movable, a wall 105′ of suction valve well 110′ may have a protrusion 530 or other type of stop formed thereon. Protrusion 530 may extend into suction valve well 110′ and may prevent or inhibit movable portion 515 from moving distally of protrusion 530.

Valve assembly 500 may transition from the active state (see FIG. 5B) to the resting state (see FIG. 5A) manually or automatically. For example, a user may actively move or release actuator 512, thereby changing valve assembly 500 to a resting state from an active state. In another example, valve assembly 500 may include a spring or other pliable material (see FIGS. 7A-7B for an example of a spring) that may be configured to cause valve assembly 500 to transition into the resting state upon the user removing force from actuator 512.

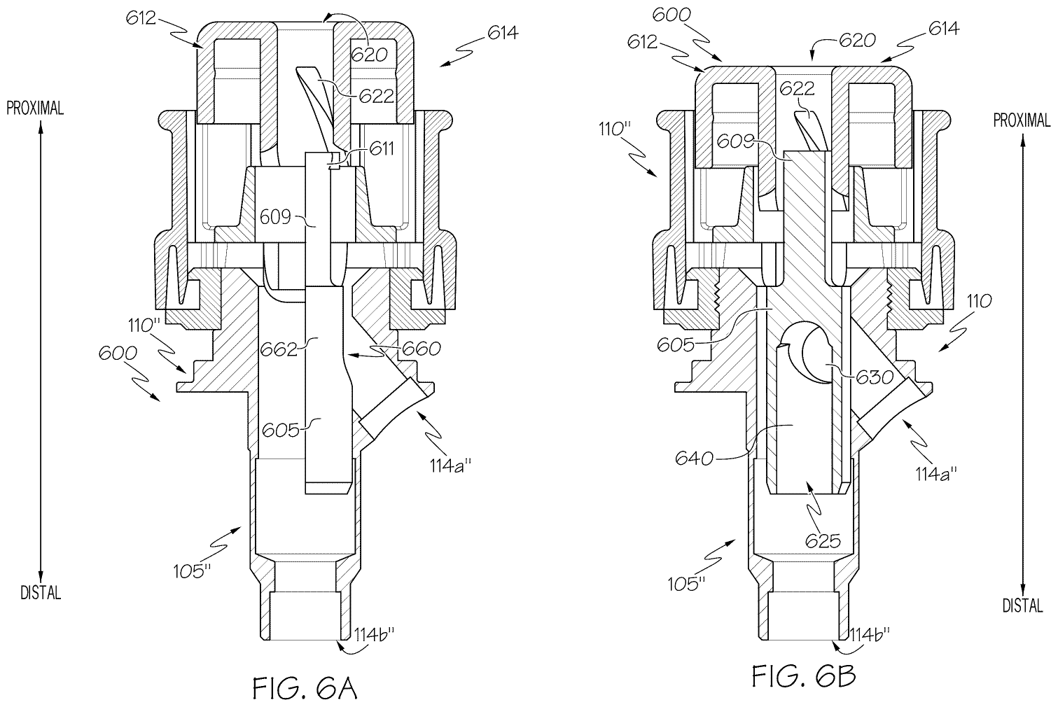

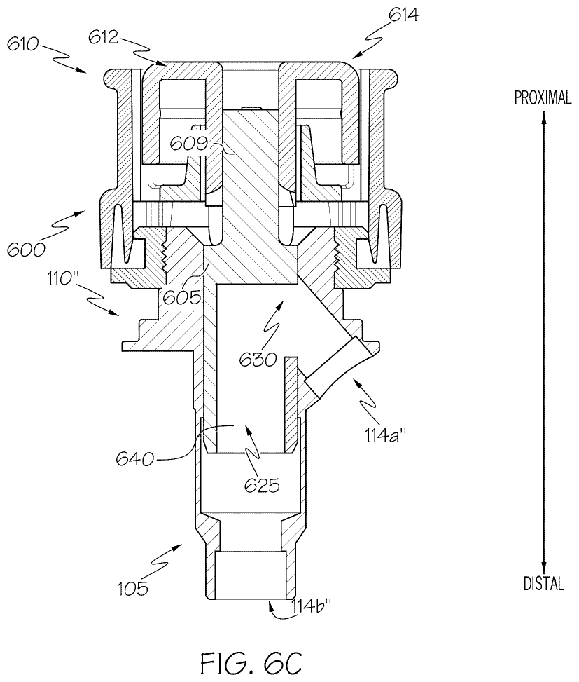

FIGS. 6A-6C show a fourth valve assembly 600 that may be configured to operate by causing a shaft 605 to rotate within suction valve well 110″. The views of FIGS. 6A-6C are all partial or complete cross-sectional views. Across FIGS. 6A-6C, the cross-section of shaft 605 is consistent, such that, as shaft 605 rotates, different portions of shaft 605 are visible in cross-section or not in cross-section. In other words, FIG. 6A shows an outside surface of shaft 605 because the cross-section of shaft 605 extends into and out of the page of FIG. 6A. Meanwhile, in FIG. 6A, the cross-section of shaft 605 is parallel to the page of FIG. 6A, such that walls of shaft 605 are shown in cross-sectional view. Fourth valve assembly 600 may have any of the features discussed in relation to valve assembly 300, valve assembly 400, valve assembly 500, the valve assembly described below, unless provided otherwise herein.

Fourth valve assembly 600 may include a shaft 605 and a proximal portion 614 (e.g., a cap) removably housed within suction valve well 110″. In contrast to the suction valve wells 110, 110′ described above, an inlet port 114a″ connected to a source of suction may be disposed on a side of suction valve well 110″, and an outlet port 114b″ connected to the working channel of device 10 may be disposed on a distal end of suction valve well 110″. Shaft 605 may be configured to rotate based on force applied to an actuator 612. Shaft 605 may include a lumen 640 extending axially through inner shaft 605. Lumen 640 may in fluid communication with an inlet aperture 630 on a side surface of shaft 605. Inlet aperture 630 may be configured to allow a flow of suction when inlet aperture 630 is substantially aligned with inlet port 114a″. A distal end of lumen 640 may include an outlet aperture 625. Although the lumen 640 is shown in cross-section in the figures, it will be appreciated that lumen 640 may be closed except for outlet aperture 625 and inlet aperture 630.

In some embodiments, shaft 605 may include one or more seals positioned thereon. For example, shaft 605 may include one or more overmolded O-rings or other types of seals. For example, one or more seals may be positioned around a diameter of shaft 605 so as to be distal of inlet aperture or surround inlet aperture 630. Alternatively, shaft 605 may be sized and shaped such that a small gap may exist that may act as a seal. Additionally or alternatively, shaft 605 may be comprised of a soft polymer, and/or thermoplastic elastomers (TPE), Liquid Silicone Rubber (LSR), etc., such that one or more seals are formed between shaft 605 and a valve well wall 105″ of suction valve well 110″.

As shown in FIG. 6A, at least a portion of shaft 605 may have an outer surface 662 that is contoured (i.e., not having a circular circumference). For example, outer surface 662 of the proximal portion of shaft 605 may include a recessed portion 660. As used herein, the term “central longitudinal axis” as it refers to shaft 605 refers to an axis that extends in a proximal/distal direction through a center of a distal portion of shaft 605 (a portion of shaft 605 not including recessed portion 660). At recessed portion 660, a distance between outer surface 662 and the central longitudinal axis may be smaller than a distance between outer surface 662 and the central longitudinal axis at a portion of shaft 605 that is just distal to recessed portion 660. Angularly, recessed portion 660 may account for approximately 90 degrees or less of a circumference of shaft 605. A centerline of recessed portion 660 may be offset from a centerline of inlet aperture 630 by approximately 90 degrees to approximately 180 degrees. For example, the centerline of recessed portion 660 may be offset from a centerline of inlet aperture 630 by at least approximately 90 degrees. As discussed in further detail below, when recessed portion 660 is aligned with inlet port 114a″, as shown in FIG. 6A, atmospheric air may flow through proximal portion 614 (e.g., through a vent opening 620), to recessed portion 660, and into inlet port 114a″. Such a configuration may provide venting of the suction source, such that suction is not delivered to the working channel of shaft 18 of medical device 10.

Actuator 612 may be, for example, a button that moves linearly in a proximal/distal (up/down) direction. Although a linear actuator 612 is described herein, it will be appreciated that actuator 612 may instead be a rotational actuator (e.g., a knob) having any of the properties of actuators 312 or 415, described above. Alternative types of actuators may also be utilized. As a button-type actuator 612 is depressed, shaft 605 may be rotated as described below.

Shaft 605 may include a proximal projection 609 that may extend into proximal portion 614 of valve 610. Proximal projection 609 may include a protrusion 611 (FIG. 6A) that is configured to pair, engage, etc. with (mate with) a notch or groove of actuator 612 (hereinafter “notch”) 622. Notch 622 may extends distally and may be curved or angled (e.g., part of a helical shape). For example, notch 622 may extend around at least a portion of a circumference of an inner surface of actuator 612. When a user applies a proximal force on actuator 612, protrusion 611 may ride along notch 622. For example, linear movement of actuator 612 may cause protrusion 611 to ride along notch 622. Because notch 622 curves around a circumference of actuator 612, as protrusion 611 rides along notch 622, shaft 605 may rotate as well as translate along an axial length of notch 622.

FIG. 6A depicts fourth valve assembly 600 in a resting state, in which suction is not delivered to the working channel of shaft 18. As shown in FIG. 6A, recessed portion 660 of shaft 605 may be aligned with inlet port 114a″. Thus, atmospheric air may flow through proximal portion 614, along recessed portion 660, and into inlet port 114a″, as described above. In the configuration of FIG. 6A, actuator 612 may not be depressed. In some examples, the configuration of FIG. 6A may be a position to which actuator 612 is biased (e.g., via a spring or similar resilient structure).

FIG. 6C depicts valve assembly 600 in an active state, in which actuator 612 may be fully depressed. FIG. 6B shows a valve assembly 600 in an intermediate state, between the states of FIG. 6A and 6C, in order to illustrate the rotational motion of shaft 605. In the active state of FIG. 6C, inlet aperture 630 may be substantially aligned (axially and rotationally) with inlet port 114a″, such that suction may flow through lumen 640, outlet aperture 625, and the working channel of shaft 18 of device 10.

Valve assembly 600 may transition from the active state (see FIG. 6A) to the resting state (see FIG. 6C) manually or automatically. For example, a user may pull actuator 612, thereby changing fourth valve assembly 600 to a resting state. In another example, valve assembly 600 may include a spring or other pliable material (see FIGS. 7A-7B for an example of a spring) that may be configured to cause fourth valve assembly 600 to move into the resting state upon the user removing a distal force from actuator 612.

FIGS. 7A-7B show a valve assembly 700 that may be configured to operate by rotating an inner shaft 705 within an outer shaft 707. Valve assembly 700 may have any of the features discussed in relation to valve assembly 300, valve assembly 400, valve assembly 500, and/or valve assembly 600, unless provided otherwise herein.

Valve assembly 700 may include suction valve well 110 and a valve 710. Valve 710 may include an outer shaft 707 and an inner shaft 705. Inner shaft 705 may be concentrically housed within outer shaft 707, and outer shaft 707 may be removably concentrically housed within suction valve well 110.

Outer shaft 707 may include one or more cuts 752 or grooves in/through a wall of outer shaft 707. Inner shaft 705 may include one or more protrusions 750 that may be received within cuts 752. Cut(s) 752 may extend around at least 90 or at least 180 degrees of a circumference of outer shaft 707. Cut(s) 752 may have a partial helical shape, such that they extend axially (proximally/distally) as well as around at least a portion of a circumference of outer shaft 707. Inner shaft 705 may include an actuator (e.g., a button) coupled to a proximal end of inner shaft 705 or formed integrally with (e.g., of a single monolithic piece with) inner shaft 705. As described below, as an operator moves actuator 712 of valve 710 distally, protrusion(s) 750 may ride along cut(s) 752, causing inner shaft 705 to rotate relative to outer shaft 707.

As shown in FIG. 7A, when valve 710 is inserted into suction valve well 110, valve 710 may be inserted until a distal end of outer shaft 707 contacts a bottom surface of wall 105 of suction valve well 110. A proximal end of inner shaft 705 may extend proximally of a proximal end of outer shaft 707 and proximally of a proximal end of an opening of suction valve well 110. Outer shaft 707 may include one or more seals or may be comprised of a material such that outer shaft 707 is rotationally fixed relative to suction valve well 110 during operation.

Inner shaft 705 may include a central lumen 740 extending axially therethrough. A distal end of lumen 740 may be open at an inlet aperture 722, which may be proximate to inlet port 114a and in fluid communication with inlet port 114a. Inner shaft 705 may also include one or more proximal apertures 725 (e.g., two proximal apertures as shown in FIG. 7A) that are in fluid communication with lumen 740. In a resting state of valve 710, proximal apertures 725 may be proximal of outer shaft 707 and/or a proximal end of suction valve well 110, such that proximal apertures 725 are open to the atmosphere. Thus, atmospheric air may enter proximal apertures 725, travel through distally through lumen 740, through inlet aperture 722, through inlet port 114b, and move toward a suction source coupled to inlet port 114b. Thus, when proximal apertures 725 are proximal of outer shaft 707, suction may be turned “off.”

Outer shaft 707 may include an outer shaft aperture 721, which may be aligned with outlet port 114b in all configurations where valve 710 is inserted into suction valve well 110. Inner shaft 705 may include a distal aperture 718 (see FIG. 7B). Distal aperture 718 may be configured to align with and be in fluid communication with outlet port 114b and outer shaft aperture 721 when valve assembly 700 is in an active state. To transition between the resting state and the active state, as discussed below, actuator 712 may be depressed, such that inner shaft 705 rotates and moves axially distally/downward. In the active state, proximal apertures 725 may be distal of a proximal-most end of outer shaft 707 and/or suction valve well 110, such that atmospheric air is prevented or inhibited from entering proximal apertures 725. With distal aperture 718 in fluid communication with outlet port 114b and proximal apertures 725 not in fluid communication with the atmosphere, suction from inlet port 114a may flow through the working channel of shaft 18, outlet port 114b, distal aperture 718, lumen 740, and inlet aperture 722.

A spring 720 may be disposed between actuator 712 and outer shaft 707 and/or suction valve well 110. As shown in FIG. 7A, when no pressure is applied to actuator 712, spring 720 may be in a natural, decompressed state. In the natural, decompressed state of spring 720, proximal apertures 725 may be proximal of outer shaft 707 and/or suction valve well 110. As shown in FIG. 7B, when a distal force is applied to actuator 712, spring 720 may be in a compressed state.

FIG. 7A depicts valve assembly 700 in a resting state. As shown in FIG. 7A, when no force is applied to actuator 712, at least one proximal aperture 725 may extend above/proximally of outer shaft 707 and/or suction valve well 110, and distal aperture 718 (not shown in FIG. 7A due to a rotation of inner shaft 705) may be in misalignment with outlet port 114b. When distal aperture 718 is in misalignment with outlet port 114b, suction may be unable to flow through outlet port 114b. Instead, as discussed above, valve 710 may be vented as atmospheric air flows toward the source of suction, through proximal aperture 725, lumen 740, inlet aperture 722, and inlet port 114a.

FIG. 7B depicts valve assembly 700 in an active state. As shown in FIG. 7B, when a user applies a distal force to actuator 712, inner shaft 705 may rotate and axially move such that distal aperture 718 moves into alignment with outlet port 114b. For example, protrusion(s) 750 may ride cut(s) 752 to rotate and axially move. Protrusion 750 is not visible in FIG. 7B due to a rotation of inner shaft 705. When distal aperture 718 is in alignment with outlet port 114b, suction from inlet port 114b may be able to flow through inlet aperture 722, lumen 740, distal aperture 718, outer shaft aperture 721, outlet port 114b, and the working channel of shaft 18.

Valve assembly 700 may transition from the active state (see FIG. 7B) to the resting state (see FIG. 7A) manually or automatically. For example, a user may press actuator 712, thereby changing valve assembly 700 to an active state. The user may pull actuator 712 to transition the valve assembly 700 to the resting state. In another example, valve assembly 700 may include spring 720 or other pliable material that may be configured to cause valve assembly 700 to automatically transition into the resting state upon the user removing distal force from actuator 712.

While principles of this disclosure are described herein with reference to illustrative examples for particular applications, it should be understood that the disclosure is not limited thereto. The systems, devices, and methods of this disclosure, may be used in any suitable medical procedure in any lumen or cavity within the body, for example, to provide suction. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A medical valve, comprising:
a shaft including a first opening on a distal end of the shaft and a second opening on a side surface of the shaft; and
an actuator configured to rotate the shaft about a longitudinal axis of the shaft in order to transition the medical valve to (a) a first configuration, in which the second opening is aligned with a port of a valve well, in order to deliver suction to a working channel of an endoscope, and
(b) a second configuration in which the second opening is misaligned with the port of the valve well.

2. The medical valve of claim 1, wherein the actuator is configured to automatically transition from the first configuration to the second configuration.

3. The medical valve of claim 1, wherein the actuator includes a rotatable knob or a rotatable lever.

4. The medical valve of claim 1, wherein the actuator includes a linear actuator configured to move in a direction parallel to the longitudinal axis of the shaft.

5. The medical valve of claim 4, wherein the linear actuator includes a button, and wherein the shaft includes a protrusion configured to engage with a groove of the button.

6. The medical valve of claim 1, wherein the shaft is an inner shaft and wherein the medical valve further comprises an outer shaft including a third opening on a distal end of the outer shaft and a fourth opening on a side surface of the outer shaft.

7. The medical valve of claim 6, wherein, in the first configuration, the second opening of the inner shaft is aligned with the fourth opening of the outer shaft.

8. The medical valve of claim 6, wherein the fourth opening is aligned with the port in the first configuration and the second configuration.

9. The medical valve of claim 6, wherein the outer shaft includes a channel on the side surface of the outer shaft, wherein the channel is aligned with the second opening of the inner shaft in the second configuration, such that the first opening of the inner shaft is in fluid communication with the channel of the outer shaft, and wherein the channel is in fluid communication with an atmosphere in at least the second configuration.

10. The medical valve of claim 6, wherein the outer shaft includes a cut or groove, and wherein the inner shaft includes a protrusion configured to engage the cut or the groove.

11. The medical valve of claim 10, wherein the actuator is fixedly coupled to the inner shaft, such that linear movement of the actuator causes the protrusion of the inner shaft to ride along the cut or the groove in order to rotate the inner shaft relative to the outer shaft.

12. The medical valve of claim 6, wherein the inner shaft includes a fifth opening, wherein, in the second configuration, the fifth opening is proximal of a proximal-most end of the outer shaft and the valve well.

13. The medical valve of claim 1, wherein an outer surface of the shaft includes a recessed portion, wherein the recessed portion is aligned with the port in the second configuration, such that the port is in fluid communication with an atmosphere in the second configuration.

14. The medical valve of claim 13, wherein the recessed portion is angularly offset from the second opening by at least approximately 90 degrees.

15. The medical valve of claim 1, further comprising an aperture configured to be closed in the first configuration and open in the second configuration.

16. A medical valve, comprising:
an inner shaft;
an outer shaft, wherein the inner shaft is concentrically received within the outer shaft; and
an actuator configured to rotate the inner shaft about a longitudinal axis of the inner shaft in order to transition the medical valve to (a) a first position, in which the medical valve is configured to deliver suction to a working channel of an endoscope, and (b) a second position, in which the medical valve inhibits delivery of suction to the working channel of the endoscope.

17. The medical valve of claim 16, wherein, in the second position, a lumen of the inner shaft is in fluid communication with an atmosphere.

18. The medical valve of claim 16, wherein the outer shaft includes a cut or groove, and wherein the inner shaft includes a protrusion configured to engage the cut or the groove.

19. A medical valve, comprising:
a threaded shaft defining a shaft lumen extending longitudinally through at least a portion of the threaded shaft; and
a rotatable body including at least one spoke, wherein the at least one spoke defines a spoke lumen;
wherein the rotatable body is configured to rotate with respect to the threaded shaft in order to selectively align the spoke lumen with a port of a suction valve well.

20. The medical valve of claim 19, wherein the port is a first port, and wherein the shaft lumen has an open distal end configured to be aligned with a second port of the suction valve well in all configurations of the medical valve.
